(19) United States
(12) Patent Application Publication
(10) Pub. No.: US 2026/0251743 A1
(43) Pub. Date: Aug. 27, 2026

(54) MAGNETIC FIELD STATE INDICATION APPARATUS FOR MAGNETIC RESONANCE IMAGING SYSTEM, AND MAGNETIC RESONANCE IMAGING SYSTEM

(71) Applicant: GE Precision Healthcare LLC, Waukesha, WI (US)

(72) Inventors: Wenwen Xiu, Beijing (CN); Yanting Huo, Beijing (CN); Xianbo Piao, Beijing (CN); Weiji Yan, Beijing (CN); Wei Li, Beijing (CN)

(21) Appl. No.: 19/538,825

(22) Filed: Feb. 12, 2026

(30) Foreign Application Priority Data
Feb. 25, 2025 (CN) 202510219522.9

(51) Int. Cl. G01R 33/54; A61B 5/00

(52) U.S. Cl. CPC G01R 33/543; A61B 5/704

(57) ABSTRACT

A magnetic field state indication apparatus for a magnetic resonance imaging system is provided. The apparatus includes: a moving portion; and a state indication portion, configured to accommodate the moving portion. The moving portion generates displacement within the state indication portion in response to changes in the magnetic field of the magnetic resonance imaging system, and the moving portion at different positions corresponds to different magnetic field states.

### CROSS REFERENCE TO RELATED APPLICATIONS

The present application claims priority and benefit of Chinese Patent Application No. CN 202510219522.9 filed on Feb. 25, 2025, which is incorporated herein by reference in its entirety.

### TECHNICAL FIELD

Embodiments of the present application relate to the technical field of medical devices, and in particular to a magnetic field state indication apparatus for a magnetic resonance imaging system, and a magnetic resonance imaging system.

### BACKGROUND

Magnetic Resonance Imaging (MRI) system is a non-invasive medical imaging technology that is widely used in clinical diagnosis and scientific research fields. The working principle of the MRI system is based on the excitation and detection of hydrogen nuclei by strong magnetic fields, thereby generating high-resolution medical images. MRI devices typically use superconducting magnets to produce stable and high-intensity magnetic fields with magnetic field strengths reaching 7 Tesla (T) or higher.

In the daily operation of the MRI system, energization and demagnetization of the magnet are essential operations. The energization process refers to gradually increasing the current of a superconducting magnet to achieve a predetermined magnetic field strength, while the demagnetization process refers to gradually reducing the current to bring the magnetic field back to zero. However, traditional energization and demagnetization operations are time-consuming. Therefore, the latest research directions include achieving one-button rapid automatic energization and automatic demagnetization of the magnet. For example, during a power outage, the magnet may be automatically demagnetized to protect the performance of the magnet, and after power restoration, when the magnet reaches the energization state, the magnet can be automatically energized.

### SUMMARY OF THE INVENTION

Since the state of a magnet may change, in order to intuitively confirm the state of the magnet, embodiments of the present application provide a magnetic field state indication apparatus and a magnetic resonance imaging system.

According to an aspect of the embodiments of the present application, a magnetic field state indication apparatus for a magnetic resonance imaging system is provided, the apparatus includes: a moving portion; and a state indication portion, configured to accommodate the moving portion. The moving portion generates displacement within the state indication portion in response to changes in the magnetic field of the magnetic resonance imaging system, and the moving portion at different positions corresponds to different magnetic field states.

According to an aspect of the embodiments of the present application, a magnetic resonance imaging system is provided, the system includes the magnetic field state indication apparatus according to the preceding aspect.

One of the beneficial effects of the embodiments of the present application is that: the moving portion may generate displacement within the state indication portion in response to changes in the magnetic field of the magnetic resonance imaging system, and the moving portion at different positions corresponds to different magnetic field states. Thereby, operators may intuitively and conveniently confirm the state of a magnet without entering a scanning room. Moreover, the indication of the state is implemented by a mechanical structure, does not rely on software, and can prevent the problem of being unable to determine the state of the magnet during a power outage. Additionally, the cost is lower and the safety and reliability are increased.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a magnetic resonance imaging system according to embodiments of the present application;

FIG. 2 is a perspective view of a magnetic field state indication apparatus according to an embodiment of the present application;

FIG. 3 is a schematic diagram illustrating indication of a magnetic field state by the magnetic field state indication apparatus according to an embodiment of the present application;

FIG. 4 is a schematic diagram illustrating indication of a magnetic field state by the magnetic field state indication apparatus according to an embodiment of the present application;

FIG. 5 is an exploded view of a magnetic field state indication apparatus according to an embodiment of the present application;

FIG. 6 is a perspective view of the magnetic field state indication apparatus according to an embodiment of the present application;

FIG. 7 is a schematic diagram illustrating indication of a magnetic field state by the magnetic field state indication apparatus according to an embodiment of the present application;

FIG. 8 is a schematic diagram illustrating indication of a magnetic field state by the magnetic field state indication apparatus according to an embodiment of the present application;

FIG. 9 is a schematic diagram of a magnetic resonance imaging system according to an embodiment of the present application;

FIG. 10 is a schematic diagram of a magnetic resonance imaging system according to an embodiment of the present application;

FIG. 11 is a schematic diagram of a magnetic resonance imaging system according to an embodiment of the present application; and

FIG. 12 is a schematic diagram illustrating scale values of a window portion according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc. are used to distinguish different elements from one another by title, but do not represent the spatial arrangement, temporal order, etc. of the elements, and the elements should not be limited by said terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies.

In the embodiments of the present application, the singular forms “a”, “the”, etc. include plural forms, and should be broadly construed as “a type of” or “a class of” rather than being limited to the meaning of “one”. Furthermore, the term “the” should be construed as including both the singular and plural forms, unless otherwise explicitly specified in the context. In addition, the term “according to” should be construed as “at least in part according to . . . ”, and the term “based on” should be construed as “at least in part based on . . . ”, unless otherwise explicitly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

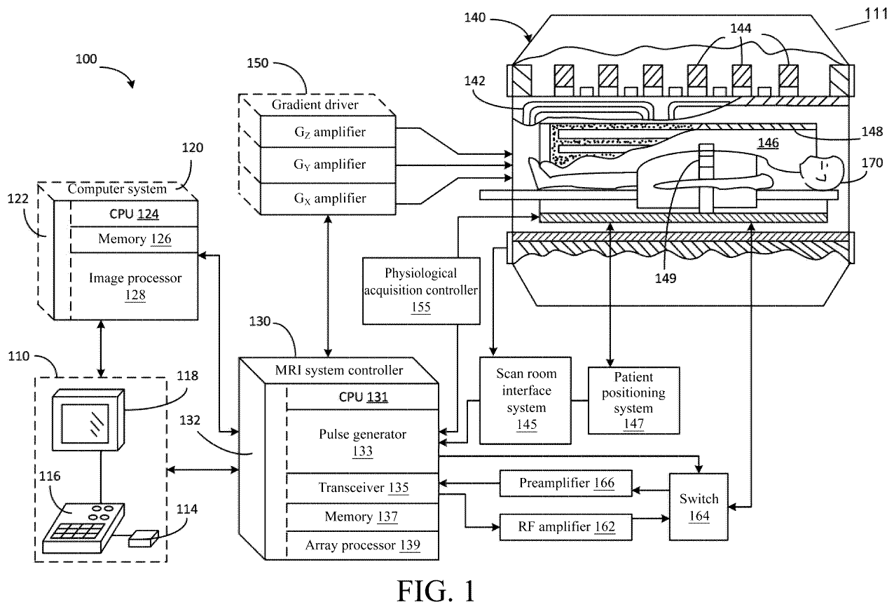

For ease of understanding, FIG. 1 shows a magnetic resonance imaging (MRI) system 100 according to some embodiments of the present invention.

The MRI system 100 includes a scanning unit 111. The scanning unit 111 is used to perform a magnetic resonance scan of a subject (e.g., a human body) 170 to generate image data of a region of interest of the subject 170, wherein the region of interest may be a pre-determined anatomical site or anatomical tissue.

Operation of the MRI system 100 is controlled by an operator workstation 110, and the operator workstation 110 includes an input device 114, a control panel 116, and a display 118. The input device 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and communicates with a computer system 120, and the computer system enables an operator to control the generation and viewing of an image on the display 118. The computer system 120 includes a plurality of components that communicate with one another by means of an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced with an image processing function implemented in the CPU 124. The computer system 120 may be connected to an archival media device, a persistent or backup memory, or a network. The computer system 120 may be coupled to and communicate with a separate MRI system controller 130.

The MRI system controller 130 includes a set of components that communicate with one another by means of an electrical and/or data connection module 132. The connection module 132 may employ a direct wired connection, a fiber optic connection, a wireless communication link, etc. The MRI system controller 130 may include a CPU 131, a pulse generator 133 that communicates with the operator workstation 110, a transceiver (or an RF transceiver) 135, a memory 137, and an array processor 139. In some embodiments, the pulse generator 133 may be integrated into the scanning unit 111 of the MRI system 100. The MRI system controller 130 may receive a command from the operator workstation 110 and is coupled to the scanning unit 111, to indicate a scan sequence that is to be executed during an MRI scan, so as to control the scanning unit 111 to execute the above-described magnetic resonance scan procedure. The “scan sequence” above refers to a combination of pulses that have specific intensities, shapes, timings, and the like applied during the execution of a magnetic resonance scan. The pulses may typically include, for example, a radio frequency pulse and a gradient pulse. The radio frequency pulses may include radio frequency excitation pulses for exciting a human body tissue, and may further include radio frequency refocusing pulses, inverse recovery pulses, etc. Typically, a plurality of scan sequences may be preset in the magnetic resonance system, so that a sequence suitable for clinical examination requirements can be selected. The clinical examination requirements may include, for example, an imaging site, an imaging function, an imaging effect, and the like.

The scanning unit 111 may include a superconducting magnet having a superconducting coil 144, a radio frequency coil assembly, and a gradient coil assembly 142. The superconducting coil 144 in a superconducting state provides a static uniform longitudinal magnetic field B₀ throughout the cylindrical imaging volume 146. The radio frequency coil assembly may include a radio frequency transmit coil and a radio frequency receive coil. The radio frequency transmit coil includes, for example, a body coil 148 or a local coil. The radio frequency receive coil includes, for example, a body coil 148 or a surface coil 149. A subject 170 to undergo a magnetic resonance scan may be positioned within the cylindrical imaging volume 146 of the scanning unit 111.

The MRI system controller 130 provides gradient waveforms to a gradient driver 150, and the gradient driver includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, to generate a magnetic field gradient used to spatially encode an MR signal during an MRI scan.

The x direction may also be referred to as a frequency encoding direction or a kₓ direction in k-space. The y direction may be referred to as a phase encoding direction or a k_{y} direction in the k-space. Gₓ may be used for frequency encoding or signal readout, and is generally referred to as a frequency encoding gradient or a readout gradient. G_{y} may be used for phase encoding, and is generally referred to as a phase encoding gradient. G_{z} may be used for slice (layer) position selection to obtain k-space data. It should be noted that a layer selection direction, a phase encoding direction, and a frequency encoding direction may be modified according to actual requirements.

In a transmit mode, a radio frequency excitation pulse sent by the pulse generator 133 may be generated by a transmit portion of the transceiver 135 (for example, including the radio frequency signal generator), and the radio frequency excitation pulse is amplified by a radio frequency power amplifier 162. Specifically, the radio frequency signal generator may generate the corresponding radio frequency excitation pulse based on the description (for example, including one or more of the amplitude, frequency, transmit power, etc.) of the radio frequency pulse in the predetermined scan sequence. The amplified radio frequency excitation pulse is provided to the radio frequency transmit coil by means of a transmit/receive switch (T/R switch) 164, and the radio frequency transmit coil in turn provides a radio frequency field B₁. The radio frequency field B₁ is substantially perpendicular to B₀ throughout the entire cylindrical imaging volume 146. The radio frequency field B₁ is used to excite stimulated nuclei in the body of the subject 170 so as to generate an MR signal. The T/R switch 164 may be controlled by a signal from the pulse generator 133 to couple, when in the transmit mode, the RF amplifier 162 to the radio frequency transmit coil and decouple, when in a receive mode, the radio frequency transmit coil from the RF amplifier 162.

In the receive mode, the MR signals emitted by excited nuclei in the body of the subject 170 may be sensed and received by an RF body coil 148 or other radio frequency receive coils and then amplified by means of a preamplifier 166.

In some embodiments, amplified MR signals are demodulated, filtered, and digitized in a receive portion of the transceiver 135.

As a non-limiting example, a transmit portion in the transceiver 135, the radio frequency power amplifier 162, and the like constitute at least a portion of a radio frequency transmit link. Furthermore, the receive portion in the transceiver 135, the preamplifier 166, the radio frequency signal demodulator (not shown), and the like constitute at least a portion of a radio frequency receive link. One or a plurality of modules/elements/assemblies of the radio frequency transmit link and the radio frequency receive link are integrated in the scanning unit 111.

The MR signals described above may be stored as raw data in the memory 137 in the MRI system controller 130, and a reconstructed magnetic resonance image may be acquired by transforming/processing the stored raw data. For example, for each image that is to be reconstructed, the data is rearranged into separate k-space data arrays, each of the separate k-space data arrays being input into the array processor 139, and the array processor 139 being operated to transform the data into an array of image data by means of a Fourier transform.

The reconstructed images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. A suitable MRI system may include more, fewer, and/or different components.

The MRI system controller 130 and the image processor 128 may separately or collectively include a computer processor and a storage medium. The storage medium records a predetermined data processing program that is to be executed by the computer processor. For example, the storage medium may store a program used to implement scan processing (for example, a scan procedure and an imaging sequence), image reconstruction, image processing, etc. For example, the storage medium may store a program used to implement the magnetic field state indication apparatus according to the embodiments of the present invention. The above storage medium may include, for example, a ROM, a floppy disk, a hard disk, an optical disk, a magneto-optical disk, a CD-ROM, or a non-volatile memory card.

Description is made below in conjunction with the embodiments.

The static magnetic field provided by the superconducting magnet described above has a very high field strength, which can reach several tesla, for example. Therefore, real-time monitoring of the magnetic field state enables timely detection and handling of abnormal situations, avoiding risks posed by high field strength to equipment or personnel. Moreover, during energization or demagnetization, if the magnetic field state can be acquired in real time through an intuitive and straightforward method, it also facilitates operators in promptly understanding the operational state of energization or demagnetization. The embodiments of the present application provide a magnetic field state indication apparatus. The apparatus includes: a moving portion; and a state indication portion, configured to accommodate the moving portion; wherein the moving portion generates displacement within the state indication portion in response to changes in the magnetic field of a magnetic resonance imaging system, and the moving portion at different positions corresponds to different magnetic field states.

Through the above embodiments, the moving portion may generate displacement within the state indication portion in response to changes in the magnetic field of the magnetic resonance imaging system, and the moving portion at different positions corresponds to different magnetic field states. Thereby, operators may intuitively and conveniently confirm the state of a magnet without entering a scanning room. Moreover, the indication of the state is implemented by a mechanical structure, does not rely on software, and can prevent the problem of being unable to determine the state of the magnet during a power outage. Additionally, the cost is lower and the safety and reliability are increased.

In the embodiments of the present application, the magnet in the magnetic resonance imaging system being in an energized state means that in this process, the field strength of the magnet gradually increases, so as to achieve enablement of the magnetic field; and the magnet being in a demagnetized state means that in this process, the field strength of the magnet gradually decreases until it completely disappears, so as to achieve disablement of the magnetic field. In the following embodiments, a magnetic field enabled state may also be replaced with a magnet energized state, a magnet energization completed state, or an energization process ended state, and a magnetic field disabled state may also be replaced with a magnet demagnetized state, a magnet demagnetization completed state, or a demagnetization process ended state.

In the embodiments of the present application, when the magnetic field is enabled (energization is completed), a position to which the moving portion moves within the state indication portion is referred to as a first position, and when the magnetic field is disabled (demagnetization is completed), a position to which the moving portion moves within the state indication portion is referred to as a second position (also referred to as an initial position). The moving portion moves between the first position and the second position when the strength of the magnetic field changes between the magnetic field enabled state and the magnetic field disabled state. Therefore, during energization, the moving portion gradually moves to the first position, and during demagnetization, the moving portion gradually moves to the second position.

In some embodiments, at least a portion of the moving portion is made of a magnetic material, which can be attracted by a magnetic field force to move. At least a portion of the moving portion may be configured in an arc shape. For example, it may be configured as a circular plate shape, an elliptical plate shape, a spherical shape, etc., to facilitate reduction of a frictional force generated when the moving portion moves inside the state indication portion. However, the embodiments of the present application are not limited thereto, and the moving portion can also be configured in other geometric shapes.

In some embodiments, to facilitate observation of the position of the moving portion, at least a portion of the moving portion may have a color different from those of a magnet housing of the magnetic resonance imaging system and the state indication portion, such as red or blue. However, the embodiments of the present application are not limited thereto. For example, the moving portion may also have a plurality of colors, and the plurality of colors are located at different positions of the moving portion and correspond to different magnetic field states, respectively, which will be described in detail in the subsequent embodiments.

In some embodiments, the state indication portion includes a housing for accommodating the moving portion. The housing may be made of a non-magnetic material, and has a shape related to the shape of the moving portion. For example, at least a portion of the edge of the housing of the state indication portion may be configured in an arc shape, so that the moving portion may be better accommodated, and the frictional force between the moving portion and the state indication portion may be further reduced. For example, the housing of the state indication portion may be set to be elliptical, semicircular, or fan-shaped, etc. The embodiments of the present application are not limited thereto, and specific examples will be described later.

The working principle of the magnetic field state indication apparatus is first explained below.

When the magnet of the magnetic resonance imaging system is being energized, a gradually increasing magnetic field is generated. As shown in FIG. 1, a magnetic field force toward the center of the superconducting magnet is generated in the superconducting magnet having the superconducting coil 144 inside the scanning unit 111. The moving portion provided in the magnetic field state indication apparatus for the magnetic resonance imaging system is attracted by the magnetic field force, and the magnetic field force gradually increases, so that the moving portion gradually moves in the state indication portion under the action of the gradually increasing magnetic field force, that is, is attracted by the magnetic field force and moves toward the center direction until energization is completed. The moving portion stops moving due to the limitation by the state indication portion and remain in a stable state, stopped at the first position.

When the magnet of the magnetic resonance imaging system is being demagnetized, the magnetic field gradually weakens, the magnetic field force gradually decreases, and an attractive force toward the center gradually disappears. Therefore, the moving portion, influenced by its own gravitational force or other acting forces (e.g., an elastic force) set in the state indication portion, gradually moves inside the state indication portion, i.e., moves in a direction away from the center until demagnetization is completed. The moving portion stops moving and returns to the initial position, i.e., the second position.

Thereby, the moving portion moves within the state indication portion under the action of the magnetic field force of the magnetic resonance imaging system and the gravitational force of the moving portion itself. The moving portion at different positions may reflect different magnetic field states, and the state of the magnet may be confirmed by confirming the position of the moving portion in the state indication portion. For example, the magnetic field of the magnetic resonance imaging system being enabled is indicated by the moving portion being located at the first position of the state indication portion, and the magnetic field of the magnetic resonance imaging system being disabled is indicated by the moving portion being located at the second position of the state indication portion. The magnet of the magnetic resonance imaging system being energized or demagnetized is indicated by the moving portion being located at a position between the first position and the second position of the state indication portion.

In some embodiments, the housing of the state indication portion is provided with a window portion. Except for the window portion, the remaining housing is a shielding portion. The window portion may be designed to be transparent, translucent, or open. The window portion is provided at a position of the housing of the state indication portion corresponding to the first position, and the moving portion is visible through the window portion when the moving portion is at the first position. That is, when the moving portion is located at the first position, an operator can observe at least a portion of the moving portion through the window portion, and when the moving portion is located at another position, the operator will not observe the moving portion due to the shielding of the shielding portion. Therefore, when at least a portion of the moving portion is visible through the window portion, it indicates that the magnetic field is enabled, and when at least a portion of the moving portion is invisible due to being shielded by the shielding portion, it indicates that the magnetic field is disabled. The size of the window portion is related to the implementation of the moving portion, and specific examples will be described later.

In some embodiments, the housing of the state indication portion is provided with a plurality of window portions, and the plurality of window portions may be designed to be transparent, translucent, or open. The plurality of window portions are provided at different positions of the housing of the state indication portion, and the moving portion is visible through the different window portions when the moving portion is located at the different positions. For example, when the moving portion is located at the first position, the operator can observe at least a portion of the moving portion through a window portion provided at the first position, and when the moving portion is located at the second position described later, the operator can observe at least a portion of the moving portion through a further window portion provided at the second position. Thus, when at least a portion of the moving portion is visible through the window portion, it indicates that the magnetic field is enabled, and when at least a portion of the moving portion is visible through the further window portion, it indicates that the magnetic field is disabled. No further examples will be provided herein.

The magnetic field state indication apparatus according to the embodiments of the present application is described below with reference to the accompanying drawings.

In some embodiments, the moving portion includes a first moving portion made of a magnetic material. When the magnetic field is enabled, the first moving portion moves to the first position of the state indication portion under the action of the magnetic field force, and when the magnetic field is disabled, the first moving portion moves to the second position of the state indication portion under the action of its own gravitational force. In addition to performing positional movement itself, the first moving portion may further indicate a magnetic field state. That is, when the first moving portion is visible through the window portion, it indicates that the magnetic field of the magnetic resonance imaging system is enabled, and when the first moving portion is invisible, it indicates that the magnetic field of the magnetic resonance imaging system is disabled.

For example, a window portion is provided at one end of the housing of the state indication portion, and except for the window portion, the remaining housing constitutes a shielding portion. When the first moving portion moves to the first position and is visible through the window portion, it indicates that the magnetic field is enabled, and when the first moving portion moves to the second position and is invisible due to being shielded by the shielding portion, it indicates that the magnetic field is disabled. When the magnetic field state indication apparatus is provided in the magnetic resonance imaging system, the first position of the state indication portion is closer to an upper side of the magnet housing accommodating the superconducting magnet than the second position of the state indication portion, and the window portion is located at a position closer to the upper side of the magnet housing than the shielding portion, so that the first moving portion returns to the second position under the action of its own gravitational force and is shielded by the shielding portion. Optionally, the first position may be closer to a magnet center than the second position, and the window portion is located closer to the magnet center than the shielding portion, or the first position and the second position are located at the same position in the vertical direction, and the window portion and the shielding portion are located at the same position in the vertical direction. Additionally, the area of the window portion needs to be set such that the area of the shielding portion is greater than the area of the first moving portion. The area of the window portion may be greater than or equal to the area of the first moving portion or less than the area of the first moving portion, and the embodiments of the present application are not limited thereto.

For example, two window portions are provided at both ends of the housing of the state indication portion. When the first moving portion moves to the first position and is visible through a window portion, it indicates that the magnetic field is enabled, and when the first moving portion moves to the second position and is visible through a further window portion, it indicates that the magnetic field is disabled. When the magnetic field state indication apparatus is provided in the magnetic resonance imaging system, the first position of the state indication portion is closer to an upper side of the magnet housing accommodating the superconducting magnet than the second position of the state indication portion, and the window portion is located at a position closer to the upper side of the magnet housing than the further window portion, so that the first moving portion returns to the second position under the action of its own gravitational force and is visible through the further window portion. Optionally, the first position may be closer to the magnet center than the second position, and the window portion is located closer to the magnet center than the further window portion, or the first position and the second position are located at the same position in the vertical direction, and the two window portions are located at the same position in the vertical direction. Additionally, the area of the window portion may be greater than or equal to the area of the first moving portion or less than the area of the first moving portion, and the embodiments of the present application are not limited thereto.

Optionally, the first moving portion may be configured to be red or blue in order to more intuitively confirm the magnetic field state through the window portion. Alternatively, in the case that an upper half portion of the first moving portion is set to be red and a lower half portion is set to be blue, and the state indication portion is provided with two window portions, when the first moving portion moves to the top end and is visible as red through a window portion, it indicates that the magnetic field is enabled, and when the first moving portion moves to the bottom end and is visible as blue through a further window portion, it indicates that the magnetic field is disabled, thereby confirming the magnetic field state.

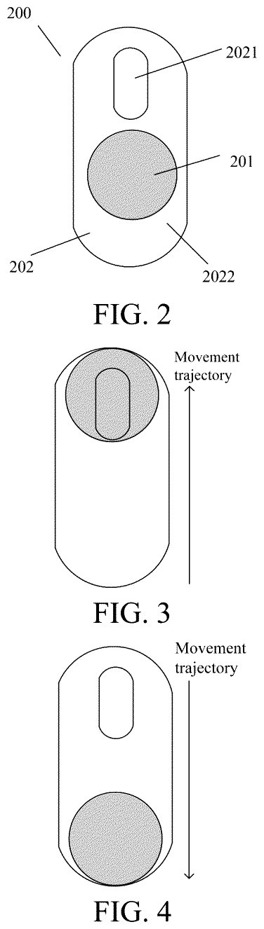

FIG. 2 is a perspective view of a magnetic field state indication apparatus according to an embodiment of the present application, and FIG. 3 and FIG. 4 are schematic diagrams illustrating indication of a magnetic field state by the magnetic field state indication apparatus according to an embodiment of the present application. As shown in FIG. 2, the magnetic field state indication apparatus 200 includes a first moving portion 201 and a state indication portion 202. The first moving portion 201 has a circular plate shape, and the state indication portion 202 has an elliptical shape (or capsule shape, or rectangular shape with rounded corners). The radius of the first moving portion 201 is slightly smaller than the semi-minor axis of the state indication portion 202, so that the first moving portion 201 can move smoothly within the state indication portion 202. A housing of the state indication portion 202 is provided with a window portion 2021 and a shielding portion 2022.

As shown in FIG. 3, during energization of a magnet, when the first moving portion 201 is attracted by a magnetic field force and moves to a first position (top end of the state indication portion), and is visible through the window portion 2021, it indicates that the magnetic field is enabled. As shown in FIG. 4, during demagnetization of the magnet, when the first moving portion 201 moves to a second position (bottom end of the state indication portion) under the action of its own gravitational force, and is invisible due to being shielded by the shielding portion 2022, it indicates that the magnetic field is disabled. Therefore, as shown in FIG. 3 and FIG. 4, the first moving portion 201 reciprocates between the first position and the second position along a linear trajectory within the state indication portion 202 during energization and demagnetization.

The shapes of the first moving portion 201 and the state indication portion 202 in FIG. 2 to FIG. 4 are merely examples, and the embodiments of the present application are not limited thereto.

In some embodiments, the moving portion includes a second moving portion made of a magnetic material and an indicator. The indicator is driven to move when the second moving portion moves. The relative position between the second moving portion and the indicator remains unchanged. For example, the second moving portion and the indicator have a connection relationship, and may be directly connected and fixed together, or the second moving portion and the indicator may be connected together through a connecting portion, such that the indicator is driven to move when the second moving portion moves. Alternatively, the second moving portion and the indicator have no connection relationship, but the indicator, during movement, contacts the second moving portion and generates an interaction force, such that the indicator is driven to move when the second moving portion moves. When the magnetic field is enabled, the second moving portion moves under the action of a magnetic force and drives the indicator to move to the first position of the state indication portion, and when the magnetic field is disabled, the second moving portion moves under the action of its own gravitational force and drives the indicator to move to the second position of the state indication portion. The second moving portion itself undergoes positional movement, but the magnetic field state is indicated by the separately provided indicator. The second moving portion itself is invisible and is not used to indicate the magnetic field state.

For example, the housing of the state indication portion is provided with a window portion, and except for the window portion, the remaining housing is a shielding portion. When the second moving portion drives the indicator to move to the first position and the indicator is visible through the window portion, it indicates that the magnetic field is enabled, and when the second moving portion drives the indicator to move to the second position and the indicator is invisible due to being shielded by the shielding portion, it indicates that the magnetic field is disabled. When the magnetic field state indication apparatus is disposed in a magnetic resonance imaging system, the first position of the state indication portion is closer to the upper side and a magnet center of a magnet housing accommodating a superconducting magnet than the second position of the state indication portion, and the window portion is located at a position closer to the upper side and the magnet center of the magnet housing than the shielding portion, so that the second moving portion drives the indicator to move to the second position under the action of its own gravitational force and is shielded by the shielding portion. The area of the window portion needs to be set such that the area of the shielding portion is greater than the area of the indicator. The area of the window portion may be greater than or equal to the area of the indicator or less than the area of the indicator, and the embodiments of the present application are not limited thereto.

For example, the housing of the state indication portion is provided with two window portions. When the second moving portion drives the indicator to move to the first position and the indicator is visible through a window portion, it indicates that the magnetic field is enabled, and when the second moving portion drives the indicator to move to the second position and the indicator is visible through a further window portion, it indicates that the magnetic field is disabled. When the magnetic field state indication apparatus is disposed in the magnetic resonance imaging system, the first position of the state indication portion is closer to the upper side and the magnet center of the magnet housing accommodating the superconducting magnet than the second position of the state indication portion, and the window portion is located at a position closer to the upper side and the magnet center of the magnet housing than the further window portion, so that the second moving portion drives the indicator to move to the second position under the action of its own gravitational force and is visible through the further window portion. The area of the window portion may be greater than or equal to the area of the indicator or less than the area of the indicator, and the embodiments of the present application are not limited thereto.

Optionally, the indicator may be set to be red or blue in order to more intuitively confirm the magnetic field state through the window portion. Alternatively, in the case that a left half portion of the indicator is set to be red and a right half portion is set to be blue, and the state indication portion is provided with two window portions, when the indicator moves to the first position and is visible as red through a window portion, it indicates that the magnetic field is enabled, and when the indicator moves to the second position and is visible as blue through a further window portion, it indicates that the magnetic field is disabled, thereby confirming the magnetic field state.

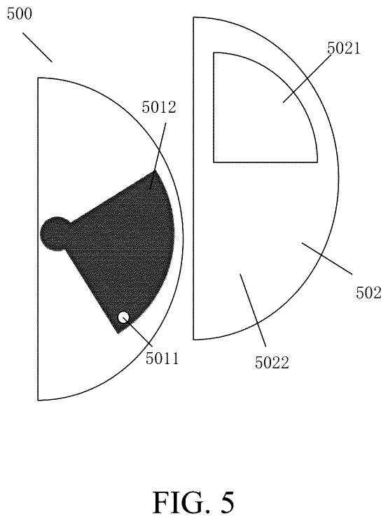

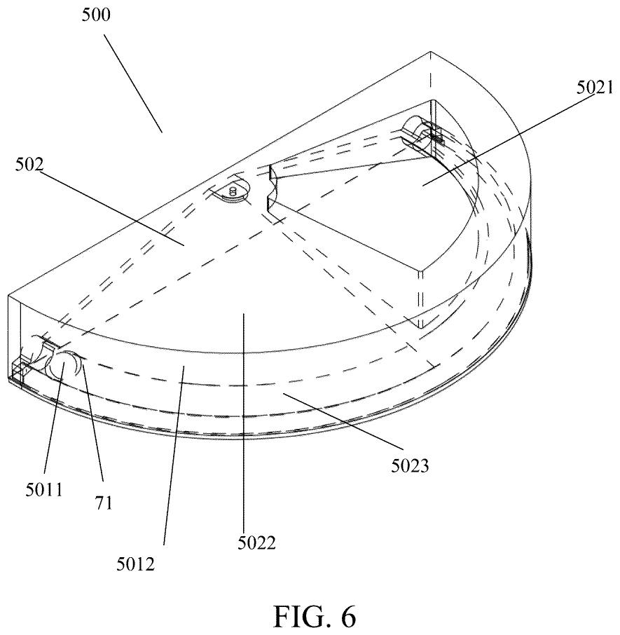

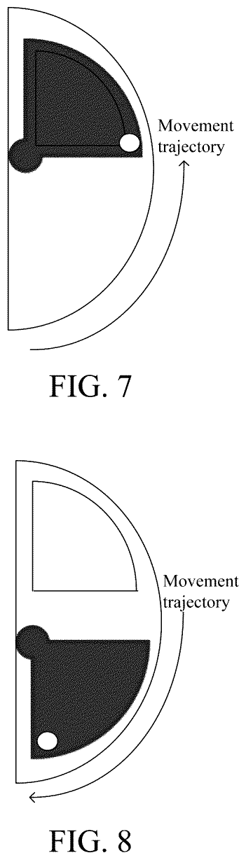

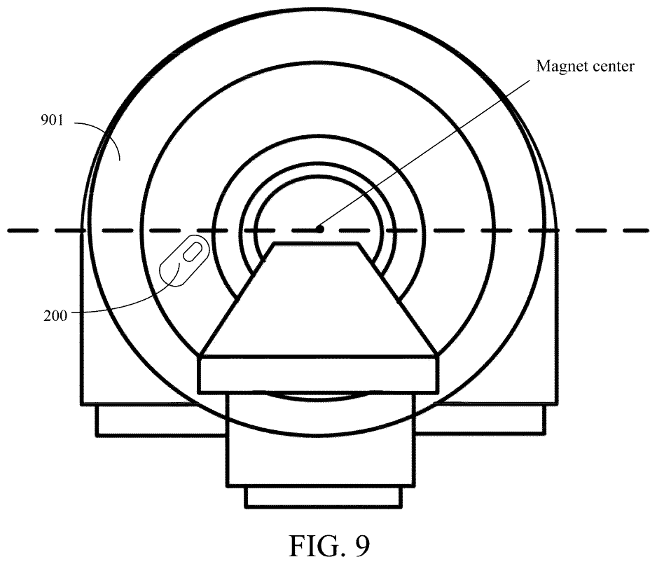

FIG. 5 is an exploded view of a magnetic field state indication apparatus according to an embodiment of the present application, FIG. 6 is a perspective view of the magnetic field state indication apparatus according to an embodiment of the present application, FIG. 7 is a schematic diagram illustrating indication of a magnetic field state by the magnetic field state indication apparatus according to an embodiment of the present application, and FIG. 8 is a schematic diagram illustrating indication of a magnetic field state by the magnetic field state indication apparatus according to an embodiment of the present application. As shown in FIG. 5 and FIG. 6, the magnetic field state indication apparatus 500 includes a second moving portion 5011, an indicator 5012 and a state indication portion 502. The state indication portion 502 is semicircular, and the indicator 5012 is sector-shaped. The sector center of the indicator 5012 is approximately at the same position as the center of the state indication portion 502, and the indicator and the state indication portion are fixed to each other, so that the indicator 5012 can rotate around the center of the semicircular state indication portion 502. The second moving portion 5011 may be configured as a sphere. A housing of the state indication portion 502 is provided with a window portion 5021 and a shielding portion 5022, and the area of the window portion 5021 needs to be set such that the area of the shielding portion 5022 is greater than the area of the indicator 5012. Additionally, a circular hole 71 may be provided at a corner of the indicator 5012. The radius of the circular hole 71 is slightly greater than that of the second moving portion 5011. The second moving portion 5011 is placed in the circular hole 71 to restrict the position of the second moving portion 5011. Thus, the second moving portion 5011, when moving, may drive the indicator 5012 to rotate. This is merely an example, and the embodiments of the present application are not limited thereto. The second moving portion 5011 may also be fixed at an edge of the indicator 5012, so that when the second moving portion moves, the indicator is driven to move. Optionally, a guide portion 5023 for guiding movement of the second moving portion 5011 is further provided inside the state indication portion 502. For example, the guide portion 5023 may be a groove rail. The guide portion 5023 is provided along an edge of the state indication portion. That is, the guide portion 5023 is provided along the inner side of an arc-shaped edge of the state indication portion, and the second moving portion 5011 is restricted to move along the guide portion 5023.

As shown in FIG. 7, when a magnet is energized, the second moving portion 5011 is attracted by the magnetic field force to move upward along the guide portion 5023, and drives the indicator 5012 to rotate to the first position (a right edge of a sector region rotates to a horizontal position), and the indicator 5012 is visible through the window portion 5021, so as to indicate that the magnetic field is enabled. As shown in FIG. 8, when the magnet is demagnetized, the second moving portion 5011 moves downward along the guide portion 5023 under the action of its own gravitational force, and drives the indicator 5012 to rotate to the second position (the right edge of the sector region rotates to an invisible vertical position), and the indicator 5012 is invisible due to being shielded by the shielding portion 5022, so as to indicate that the magnetic field is disabled. Therefore, as shown in FIG. 7 and FIG. 8, during energization and demagnetization, the second moving portion 5011 reciprocates between the first position and the second position along an arc-shaped trajectory (e.g., the guide portion 5023 or the arc-shaped edge of the state indication portion) in the state indication portion 502. Since the second moving portion moves along the arc-shaped trajectory, the stroke of the second moving portion is longer, thereby facilitating precise quantitative indication of the energization or demagnetization process. Furthermore, compared with the magnetic field state indication apparatus 200 in FIG. 2, the magnetic field state indication apparatus 500 is disposed at more flexible positions on the magnet housing, for example, is supported to be disposed on an upper half portion of the magnet housing.

The shapes and positional relationships of the second moving portion, the indicator, and the state indication portion in FIG. 5 to FIG. 8 are merely examples, and the embodiments of the present application are not limited thereto. For example, in FIG. 2, an indicator may also be provided. The first moving portion, when moving, may drive the indicator to move, and the magnetic field state is indicated by whether the indicator is visible. No further examples will be provided herein.

In some embodiments, since energization and demagnetization are processes, to quantitatively confirm the progress of energization and demagnetization, indication scales may also be provided at the edge of the window portion of the state indication portion. For example, the scale values may be determined based on parameters such as the length of the window portion, the magnitude of a friction force generated inside the moving portion and the state indication portion, the installation position and inclination of the magnetic field state indication apparatus, and the magnitude of the magnetic field force. The physical quantity of the scale values may be the progress percentage of energization and demagnetization, or the absolute magnitude or relative magnitude of the field strength, etc. The embodiments of the present application are not limited thereto. FIG. 12 is a schematic diagram illustrating scale values of the window portion according to an embodiment of the present application. As shown in FIG. 12, for the two embodiments of FIG. 2 and FIG. 5, indication scales may be provided at an edge of the window portion. The physical quantity of the scale values is the progress percentage of energization and demagnetization. Accordingly, when the moving portion gradually appears in the window portion or gradually recedes from the window portion, the current progress of energization or demagnetization may be determined according to the scale of the window portion with which the edge of the moving portion is aligned, thereby quantitatively confirming the magnetic field state.

In the above examples, the movement of the moving portion to the second position under the action of its own gravitational force during demagnetization is used as an illustrative example. However, the embodiments of the present application are not limited thereto. For example, when the magnetic field force gradually decreases, the moving portion may also move to the second position under the action of an elastic force provided by an elastic structure disposed inside the state indication portion. No further examples will be provided herein.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

Through the above embodiments, the moving portion may generate displacement within the state indication portion in response to changes in the magnetic field of the magnetic resonance imaging system, and the moving portion at different positions corresponds to different magnetic field states. Thereby, operators may intuitively and conveniently confirm the state of a magnet without entering a scanning room. Moreover, the indication of the state is implemented by a mechanical structure, does not rely on software, and can prevent the problem of being unable to determine the state of the magnet during a power outage. Additionally, the cost is lower and the safety and reliability are increased.

Embodiments of the present application further provide a magnetic resonance imaging system. The configuration of the magnetic resonance imaging system is as shown in FIG. 1, and similarities are not repeated here.

In some embodiments, what differs from the foregoing magnetic resonance imaging system in FIG. 1 is that the system may further include the magnetic field state indication apparatus described in the aforementioned embodiments. The magnetic field state indication apparatus may be provided on a magnet housing of the magnetic resonance imaging system, and at least a superconducting magnet is accommodated inside the magnet housing. For example, the scanning unit 111 in FIG. 1 may be partially or integrally accommodated in the magnet housing. For example, the magnetic field state indication apparatus may be embedded in a front panel of the magnet housing, or attached to the front panel of the magnet housing. The embodiments of the present application are not limited thereto. At least a portion of the magnetic field state indication apparatus is disposed on a lower half portion of the magnet housing of the magnetic resonance imaging system. For example, the magnetic field state indication apparatus may be disposed on the lower half portion of the magnet housing (i.e., a portion below a magnet center); or a portion of the magnetic field state indication apparatus is disposed on the lower half portion of the magnet housing and the other portion is disposed on an upper half portion of the magnet housing (i.e., a portion above the magnet center); or the magnetic field state indication apparatus may be disposed on the upper half portion of the magnet housing and at a position close to the horizontal line of the magnet center. The embodiments of the present application are not limited thereto.

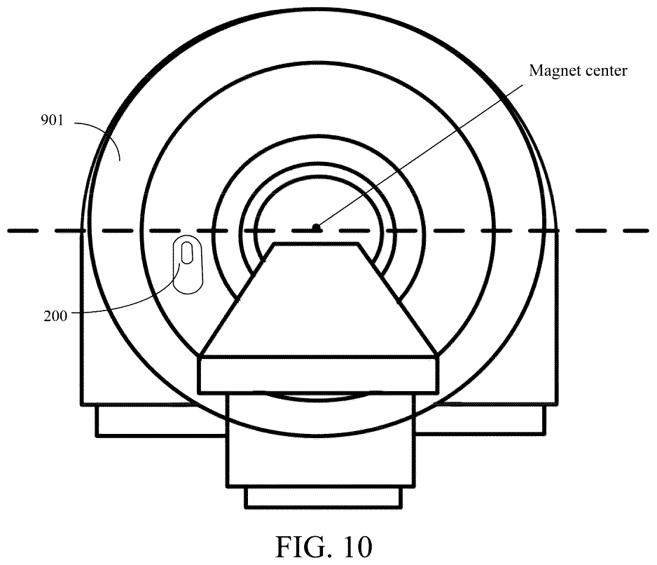

FIG. 9 and FIG. 10 are schematic diagrams of a magnetic resonance imaging system according to an embodiment of the present application. As shown in FIG. 9, for the magnetic field state indication apparatus 200 shown in FIG. 2, it may be disposed obliquely on an annular casing 901 of the magnet housing, where the window portion is close to the magnet center (center of a circle) and is closer to the upper side; and the shielding portion is away from the magnet center and is closer to the lower side. The magnetic field state indication apparatus may be disposed on the lower half portion of the annular casing of the magnet housing. However, the embodiments of the present application are not limited thereto. A portion of the magnetic field state indication apparatus 200 may be disposed on the lower half portion of the annular casing of the magnet housing, and the other portion of the magnetic field state indication apparatus 200 may be disposed on the upper half portion of the annular casing of the magnet housing; or the magnetic field state indication apparatus 200 may be disposed on the upper half portion of the annular casing and close to the horizontal line of the center of the annular casing of the magnet housing. The embodiments of the present application are not limited thereto. As shown in FIG. 10, the magnetic field state indication apparatus 200 may be vertically disposed on the annular casing 901 of the magnet housing, where the window portion is located on the upper side and the shielding portion is located on the lower side, and the magnetic field state indication apparatus 200 is disposed on the lower half portion of the annular casing 901. No further examples will be provided herein.

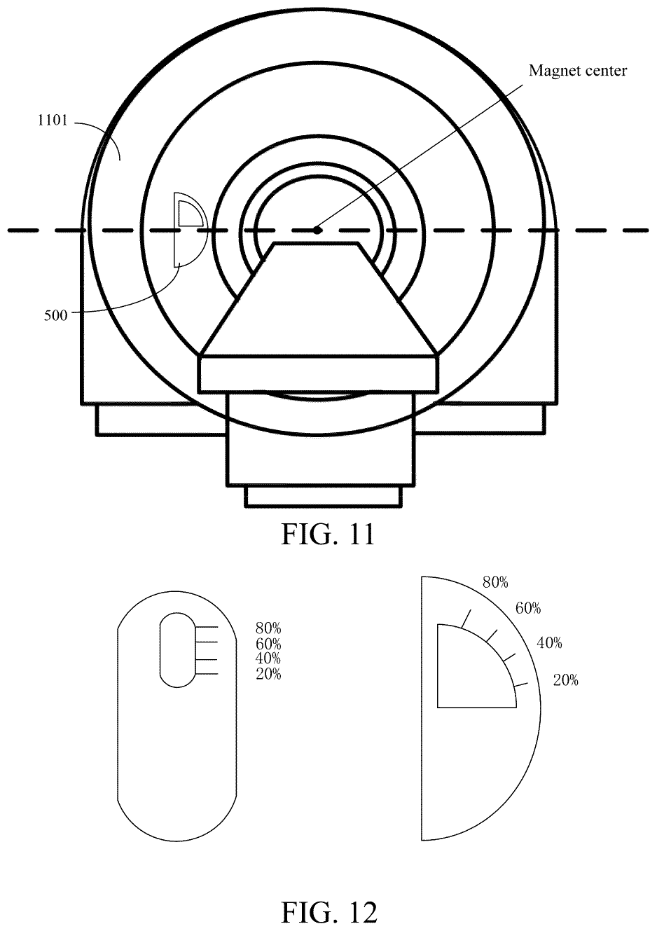

FIG. 11 is a schematic diagram of a magnetic resonance imaging system according to an embodiment of the present application. As shown in FIG. 11, for the magnetic field state indication apparatus 500 shown in FIG. 5, the window portion is located on the upper side, and the shielding portion is located on the lower side, a portion of the magnetic field state indication apparatus is disposed on the lower half portion of an annular casing 1101 of the magnet housing, and the other portion of the magnetic field state indication apparatus is disposed on the upper half portion of the annular casing 1101 of the magnet housing. However, the embodiments of the present application are not limited thereto. The magnetic field state indication apparatus 500 may also be provided on the lower half portion of the annular casing of the magnet housing. No further examples will be provided herein.

The foregoing positions and angles of the magnetic field state indication apparatus are merely examples. The present application is not limited thereto.

All articles and references disclosed, including patent applications and publications, are incorporated herein by reference for all purposes. The term “consisting essentially of” to describe a combination shall include identified elements, ingredients, components or steps, and other elements, ingredients, components or steps that do not materially affect the basic and novel characteristics of the combination. The use of the terms “comprising” or “including” to describe combinations of elements, ingredients, components or steps herein also contemplates embodiments that consist essentially of those elements, ingredients, components or steps. The use of the term “may” herein is intended to describe that any attribute included in “may” is optional. A plurality of elements, ingredients, components or steps can be provided by a single integrated element, ingredient, component or step. Alternatively, a single integrated element, ingredient, component or step may be divided into a plurality of separate elements, ingredients, components or steps. The disclosure of “a” or “one” to describe an element, ingredient, component or step is not intended to exclude other elements, ingredients, components or steps.

The embodiments in the present specification are described in a progressive manner, with each embodiment focusing on differences from other embodiments. The same or similar portions between the embodiments can be referred to mutually. The above embodiments are merely used to illustrate the technical concept and features of the present application, and are intended to enable those skilled in the art to understand the content of the present application and implement it accordingly, and are not intended to limit the scope of protection of the present application. Any equivalent changes or modifications made according to the spirit of the present application shall fall within the scope of protection of the present application.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A magnetic field state indication apparatus for a magnetic resonance imaging system, characterized by comprising:
a moving portion; and
a state indication portion, configured to accommodate the moving portion;
wherein the moving portion generates displacement within the state indication portion in response to changes in the magnetic field of the magnetic resonance imaging system, and the moving portion at different positions corresponds to different magnetic field states.

2. The apparatus according to claim 1, wherein the moving portion moves within the state indication portion under the action of a magnetic field force of the magnetic resonance imaging system and a gravitational force of the moving portion itself.

3. The apparatus according to claim 1, wherein the magnetic field of the magnetic resonance imaging system being enabled is indicated by the moving portion being located at a first position of the state indication portion, and the magnetic field of the magnetic resonance imaging system being disabled is indicated by the moving portion being located at a second position of the state indication portion.

4. The apparatus according to claim 3, wherein the moving portion moves between the first position and the second position when the strength of the magnetic field changes between a magnetic field enabled state and a magnetic field disabled state.

5. The apparatus according to claim 3, wherein the state indication portion has a window portion through which the moving portion is visible when located at the first position.

6. The apparatus according to claim 5, wherein when at least a portion of the moving portion is visible, it indicates that the magnetic field of the magnetic resonance imaging system is enabled, and when at least a portion of the moving portion is invisible, it indicates that the magnetic field of the magnetic resonance imaging system is disabled.

7. The apparatus according to claim 5, wherein the moving portion comprises a first moving portion made of a magnetic material, and when the first moving portion is visible through the window portion, it indicates that the magnetic field of the magnetic resonance imaging system is enabled, and when the first moving portion is invisible, it indicates that the magnetic field of the magnetic resonance imaging system is disabled;
or, the moving portion comprises a second moving portion made of a magnetic material and an indicator, the second moving portion, when moving, drives the indicator to move, and when the indicator is visible through the window portion, it indicates that the magnetic field of the magnetic resonance imaging system is enabled, and when the indicator is invisible, it indicates that the magnetic field of the magnetic resonance imaging system is disabled.

8. The apparatus according to claim 1, wherein a guide portion for guiding movement of the moving portion is further provided inside the state indication portion.

9. The apparatus according to claim 8, wherein the guide portion is disposed along an edge of the state indication portion.

10. The apparatus according to claim 1, wherein at least a portion of an edge of the state indication portion is configured in an arc shape.

11. The apparatus according to claim 1, wherein the magnetic field state indication apparatus is disposed on a magnet housing of the magnetic resonance imaging system, and a first position of the state indication portion is closer to an upper side of the magnet housing or a magnet center of the magnet housing than a second position of the state indication portion, wherein at least a superconducting magnet is accommodated inside the magnet housing.

12. The apparatus according to claim 11, wherein at least a portion of the magnetic field state indication apparatus is disposed in a lower half portion of the magnet housing of the magnetic resonance imaging system, or at least a portion of the magnetic field state indication apparatus is disposed in an upper half portion of the magnet housing and at a position proximate to a horizontal centerline of the magnet housing.

13. A magnetic resonance imaging system, characterized by comprising:
the magnetic field state indication apparatus according to claim 1.
